(19) United States
(12) Patent Application Publication
(10) Pub. No.: US 2007/0082047 A1
(43) Pub. Date: Apr. 12, 2007

(54) Solid preparation

(76) Inventors: Masae Sugaya, Osaka-shi (JP); Hiroyoshi Koyama, Osaka-shi (JP); Naoru Hamaguchi, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/578,136

(22) PCT Filed: Nov. 4, 2004
(86) PCT No.: PCT/JP04/16701

§ 371 Date: May 3, 2006

(30) Foreign Application Priority Data
Nov. 7, 2003 (JP) 2003-378470

(51) Int. Cl. A61K 31/4439; A61K 9/20

(52) U.S. Cl. 424/464; 514/338

(57) ABSTRACT

A chewable tablet comprising a group which contains an acid-labile active ingredient and at least one basic substance selected from alkaline earth metal carbonate, metal oxide and metal hydroxide, and a group which does not contain an acid-labile active ingredient and contains at least one ingredient selected from alkaline earth metal carbonate, metal oxide and metal hydroxide, wherein said chewable tablet is capable of rapidly neutralizing gastric acid and is preferably not enteric-coated, is provided.

### TECHNICAL FIELD

The present invention relates to a solid preparation, more particularly, a pharmaceutical solid preparation containing an acid-labile active ingredient, in particular an acid-labile active ingredient useful as an anti-tumor agent such as a benzimidazole compound.

### BACKGROUND ART

Since benzimidazole proton pump inhibitor (hereinafter, referred to as PPI) compounds such as lansoprazole, omeprazole and rabeprazole and imidazopyridine PPI compounds such as tenatoprazole have gastric acid secretion inhibiting activity, stomach mucosa defending activity and the like, they are widely used as a drug for treating peptic ulcer.

However, these PPI compounds are unstable and sensitive to humidity, temperature and light. They are particularly sensitive to acid and aqueous solutions or suspensions of them become unstable extremely as their pH becomes lower.

The PPI compounds are also more unstable in a preparation (e.g. tablet, powder, fine granule, capsule, etc.) than be alone because they strongly interact with other ingredients of the preparation formulation. Therefore, a change in the color of the preparation or degradation of the active ingredient is observed in producing or keeping the preparation.

In order to stabilize PPI compounds in a preparation, JP-A 62-277322 discloses an enteric granule, an enteric fine granule and the like obtained by blending a stabilizer comprising a basic inorganic salt such as magnesium and/or calcium for a pharmaceutical solid composition and then coating with an enteric coating agent.

In order to obtain such an enteric preparation, however, a step of producing a fine granule or a granule containing PPI such as a benzimidazole PPI compound and then enteric-coating it is needed. In addition, it takes substantial time to accomplish digestion of the enteric film in an alimentary canal and then absorption of a drug after administering such an enteric preparation and therefore, rapid pharmacological effect at an initial stage after administration is hardly expected.

U.S. Pat. No. 5,840,737 and WO 00/26185 disclose a solution, suspension, tablet, capsule and the like of omeprazole or lansoprazole in combination with alkali metal bicarbonate without an enteric-coating.

### DISCLOSURE OF INVENTION

An object of the present invention is to provide a stable pharmaceutical solid preparation, in particular a stable chewable tablet, which contains an acid-labile active ingredient including PPI such as a benzimidazole compound and an imidazopyridine compound having proton pump inhibitor (PPI) activity.

More particularly, an object of the present invention is to provide a solid preparation which can rapidly neutralize acid in stomach and which is not enteric-coated in order to solve the aforementioned problems in PPI such as benzimidazole PPI compounds, wherein said solid preparation is a chewable tablet, which can be taken easily even by children or the elderly, capable of rapidly eliciting the pharmacological effect of an active ingredient.

The present inventors studied intensively to provide a solid preparation containing an acid-labile active ingredient such as a benzimidazole PPI compound or an imidazopyridine PPI compound and capable of rapidly neutralizing acid in stomach, and as a result, found that a chewable agent comprising PPI in combination with alkaline earth metal carbonate, metal oxide and metal hydroxide had improved storage stability and could rapidly elicit the pharmacological effect. Based on this finding, the present inventors further studied and as a result, completed the present invention.

That is, the present invention relates to:

(1) a chewable tablet, comprising an acid-labile active ingredient and at least one ingredient selected from alkaline earth metal carbonate, metal oxide and metal hydroxide;

(2) the chewable tablet according to the above (1), further comprising a highly water-soluble basic additive;

(3) the chewable tablet according to the above (1), wherein its ingredients are divided into a group which contains an acid-labile active ingredient and at least one basic substance selected from alkaline earth metal carbonate, metal oxide and metal hydroxide, and a group which does not contain an acid-labile active ingredient and contains at least one ingredient selected from alkaline earth metal carbonate, metal oxide and metal hydroxide;

(4) the chewable tablet according to the above (3), wherein the group which does not contain an acid-labile active ingredient further contains a highly water-soluble basic additive;

(5) the chewable tablet according to the above (1), which is not enteric-coated;

(6) the chewable tablet according to the above (1), wherein the acid-labile active ingredient is a proton pump inhibitor (PPI);

(7) the chewable tablet according to the above (6), wherein the PPI is a benzimidazole compound;

(8) the chewable tablet according to the above (7), wherein the benzimidazole compound is a compound represented by the formula (I): 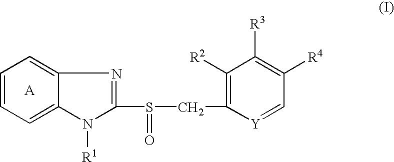
wherein ring A represents an optionally substituted benzene ring, R¹ represents hydrogen, optionally substituted aralkyl, acyl or acyloxy, R², R³ and R⁴ may be the same or different and each represents hydrogen, optionally substituted alkyl, optionally substituted alkoxy or optionally substituted amino, and Y represents nitrogen or CH, or an optically active isomer thereof or a salt thereof;

(9) the chewable tablet according to the above (7), wherein the benzimidazole compound is lansoprazole, omeprazole, rabeprazole, pantoprazole, ilaprazole or an optically active isomer thereof or a salt thereof;

(10) the chewable tablet according to the above (6), wherein the PPI is an imidazopyridine compound;

(11) the chewable tablet according to the above (10), wherein the imidazopyridine compound is tenatoprazole or an optically active isomer thereof, or a salt thereof;

(12) the chewable tablet according to the above (1), wherein the alkaline earth metal carbonate is calcium carbonate or magnesium carbonate;

(13) the chewable tablet according to the above (1), wherein the metal oxide and the metal hydroxide are in the form of a 1% (W/W) aqueous solution or suspension with pH 8.0 or higher;

(14) the chewable tablet according to the above (1), containing at least one metal oxide selected from the group consisting of magnesium oxide, magnesium silicate, dry aluminum hydroxide gel and magnesium aluminometasilicate;

(15) the chewable tablet according to the above (1), containing at least one metal hydroxide selected from the group consisting of magnesium hydroxide, aluminum hydroxide, synthetic hydrotalcite, a coprecipitate of aluminum hydroxide and magnesium hydroxide, a coprecipitate of aluminum hydroxide, magnesium carbonate and calcium carbonate, and a coprecipitate of aluminum hydroxide and sodium hydrogen carbonate;

(16) the chewable tablet according to the above (2) or (4), wherein the highly water-soluble basic additive is trometamol, disodium succinate, sodium hydrogen phosphate, trisodium phosphate, dipotassium phosphate or L-arginine;

(17) the chewable tablet according to the above (1), containing calcium carbonate;

(18) the chewable tablet according to the above (1), containing magnesium oxide;

(19) the chewable tablet according to the above (1), containing magnesium hydroxide;

(20) the chewable tablet according to the above (1), containing calcium carbonate, magnesium oxide and/or magnesium hydroxide; and

(21) the chewable tablet according to the above (7), containing 0.1 to 1500 parts by weight of at least one ingredient selected from alkaline earth metal carbonate, metal oxide and metal hydroxide per 1 part by weight of the benzimidazole compound.

In addition, the present invention relates to:

(22) a chewable tablet, comprising a group which contains an acid-labile active ingredient and at least one basic substance selected from alkaline earth metal carbonate, metal oxide and metal hydroxide, and a group which does not contain an acid-labile active ingredient and contains at least one ingredient selected from alkaline earth metal carbonate, metal oxide and metal hydroxide;

(23) the chewable tablet according to the above (22), which is not enteric-coated;

(24) the chewable tablet according to the above (22), wherein the acid-labile active ingredient is a proton pump inhibitor (PPI);

(25) the chewable tablet according to the above (24), wherein the PPI is a benzimidazole compound;

(26) the chewable tablet according to the above (25), wherein the benzimidazole compound is a compound represented by the formula (I): 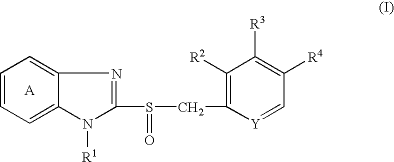
wherein ring A represents an optionally substituted benzene ring, R¹ represents hydrogen, optionally substituted aralkyl, acyl or acyloxy, R², R³ and R⁴ may be the same or different and each represents hydrogen, optionally substituted alkyl, optionally substituted alkoxy or optionally substituted amino, and Y represents nitrogen or CH, or an optically active isomer thereof or a salt thereof;

(27) the chewable tablet according to the above (25), wherein the benzimidazole compound is lansoprazole, omeprazole, rabeprazole, pantoprazole, ilaprazole or an optically active isomer thereof or a salt thereof;

(28) the chewable tablet according to the above (24), wherein the PPI is an imidazopyridine compound;

(29) the chewable tablet according to the above (28), wherein the imidazopyridine compound is tenatoprazole or an optically active isomer thereof or a salt thereof;

(30) the chewable tablet according to the above (22), wherein the alkaline earth metal carbonate is calcium carbonate or magnesium carbonate;

(31) the chewable tablet according to the above (22), wherein the metal oxide and the metal hydroxide are in the form of a 1% (W/W) aqueous solution or suspension with pH 8.0 or higher;

(32) the chewable tablet according to the above (22), containing at least one metal oxide selected from the group consisting of magnesium oxide, magnesium silicate, dry aluminum hydroxide gel and magnesium aluminometasilicate;

(33) the chewable tablet according to the above (22), containing at least one metal hydroxide selected from the group consisting of magnesium hydroxide, aluminum hydroxide, synthetic hydrotalcite, a coprecipitate of aluminum hydroxide and magnesium hydroxide, a coprecipitate of aluminum hydroxide, magnesium carbonate and calcium carbonate, and a coprecipitate of aluminum hydroxide and sodium hydrogen carbonate;

(34) the chewable tablet according to the above (22), wherein the alkaline earth metal carbonate is calcium carbonate;

(35) the chewable tablet according to the above (22), wherein the metal oxide is magnesium oxide;

(36) the chewable tablet according to the above (22), wherein the metal hydroxide is magnesium hydroxide;

(37) the chewable tablet according to the above (25), containing 0.1 to 1500 parts by weight of at least one ingredient selected from alkaline earth metal carbonate, metal oxide and metal hydroxide per 1 part by weight of the benzimidazole compound;

(38) a chewable tablet, comprising a group which contains an acid-labile active ingredient and alkaline earth metal carbonate, and a group which does not contain an acid-labile active ingredient and contains at least one ingredient selected from metal oxide and metal hydroxide;

(39) the chewable tablet according to the above (38), wherein the alkaline earth metal carbonate is calcium carbonate, the metal oxide is magnesium oxide and the metal hydroxide is magnesium hydroxide;

(40) a chewable tablet, comprising a group which contains 0.001 to 0.3 parts by weight of lansoprazole or an optically active isomer thereof or a salt thereof per 1 part by weight of the tablet and 0.2 to 200 parts by weight of calcium carbonate per 1 part by weight of lansoprazole or an optically active isomer thereof or a salt thereof, and a group which does not contain lansoprazole or an optically active isomer thereof or a salt thereof and contains total 0.2 to 200 parts by weight of magnesium oxide and magnesium hydroxide per 1 part by weight of lansoprazole or an optically active isomer thereof or a salt thereof;

(41) the chewable tablet according to the above (40), wherein the weight ratio between magnesium oxide and magnesium hydroxide is 1:0.2 to 1:50.

Although the pharmaceutical solid preparation of the present invention contains an acid-labile active ingredient, for example, a benzimidazole PPI compound or an imidazopyridine PPI compound, it does not need to be enteric-coated, and therefore it can be produced by a simple process. In addition, since the initial dissolution rate of an active ingredient from a preparation without an enteric-coating is higher than that of a preparation with an enteric-coating, the solid preparation of the present invention can reduce the time required to elicit the pharmacological activity. Further, by a combination with alkaline earth metal carbonate, metal oxide and/or metal hydroxide, or a highly water-soluble basic additive, the solid preparation of the present invention can neutralize the inside of stomach to stabilize the active ingredient. In addition, the chewable tablet of the present invention has the advantage that children or the elderly easily take it and it can be taken without water by chewing.

### DETAILED EXPLANATION OF THE INVENTION

The acid-labile active ingredient used in the present invention is not particularly limited and may be any active ingredient which becomes unstable when exposed to gastric acid. The acid-labile active ingredient includes PPI, erythromycin antibacterial compounds, and antiphlogistic enzymatic agents such as serrapeptase and semi-alkaline proteinase. In particular, PPI is suitable for the present invention. Such PPI includes benzimidazole compounds such as lansoprazole, imidazopyridine compounds such as tenatoprazole, and analogous compounds thereof. Embodiments of the benzimidazole compound will be described below. However, the acid-labile active ingredient used in the present invention is not limited to the above-mentioned compounds and other acid-labile active ingredients can be used similarly.

The benzimidazole PPI compound used in the present invention includes a compound represented by the formula (I): 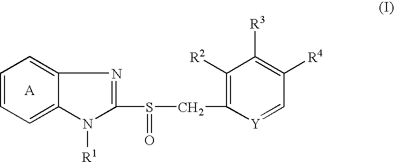
wherein ring A represents an optionally substituted benzene ring, R¹ represents hydrogen, optionally substituted aralkyl, acyl or acyloxy, R², R³ and R⁴ may be the same or different and each represents hydrogen, optionally substituted alkyl, optionally substituted alkoxy or optionally substituted amino, and Y represents nitrogen or CH, or a salt thereof.

Preferred is a compound of the formula (I) wherein ring A is a benzene ring optionally substituted with a substituent or substituents selected form halogen, optionally halogenated C₁₋₄ alkyl, optionally halogenated C₁₋₄ alkoxy and a 5- or 6-membered heterocyclic group, R¹ is hydrogen, R² is C₁₋₆ alkyl, C₁₋₆ alkoxy, C₁₋₆ alkoxy-C₁₋₆ alkoxy or a di-C₁₋₆ alkylamino, R³ is hydrogen, C₁₋₆ alkoxy-C₁₋₆ alkoxy or optionally halogenated C₁₋₆ alkoxy, R⁴ is hydrogen or C₁₋₆ alkyl, and Y is nitrogen.

Particularly preferred is a compound represented by the formula (Ia): 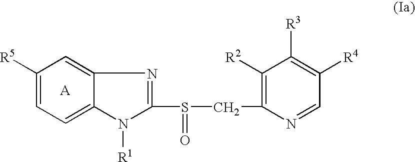
wherein R¹ represents hydrogen, R² represents C₁₋₃ alkyl or C₁₋₃ alkoxy, R³ represents C₁₋₃ alkoxy which may be halogenated or may be substituted with C₁₋₃ alkoxy, R⁴ represents hydrogen or C₁₋₃ alkyl, and R⁵ represents hydrogen, optionally halogenated C₁₋₃ alkoxy or pyrrolyl (e.g. 1-, 2- or 3-pyrrolyl).

Particularly preferred is a compound of the formula (Ia) wherein R¹ is hydrogen, R² is C₁₋₃ alkyl, R³ is optionally halogenated C₁₋₃ alkoxy, R⁴ is hydrogen, and R⁵ is hydrogen or optically halogenated C₁₋₃ alkoxy.

A “substituent” for the “optionally substituted benzene ring” represented by ring A in the compound represented by the aforementioned formula (I) [hereinafter, referred to as Compound (I)] includes halogen, cyano, nitro, optionally substituted alkyl, hydroxyl, optionally substituted alkoxy, aryl, aryloxy, carboxy, acyl, acyloxy, and 5- to 10-membered heterocyclic groups. The optionally substituted benzene ring may be substituted with about 1 to 3 substituents. When the number of substituents is 2 or more, they may be the same as or different from each other. Among the above-mentioned substituents, halogen, optionally substituted alkyl and optionally substituted alkoxy are preferable.

The halogen includes fluorine, chlorine and bromine. Inter aria, fluorine is preferable.

The “alkyl” of the “optionally substituted alkyl” includes C₁₋₇ alkyl (e.g. methyl, ethyl, propyl, isopropyl, butyl, isobutyl, sec-butyl, tert-butyl, pentyl, hexyl, heptyl, etc.). A “substituent” for the “optionally substituted alkyl” includes halogen, hydroxyl, C₁₋₆ alkoxy (e.g. methoxy, ethoxy, propoxy, butoxy, etc.), C₁₋₆ alkoxy-carbonyl (e.g. methoxycarbonyl, ethoxycarbonyl, propoxycarbonyl, etc.) and carbamoyl. The optionally substituted alkyl may be substituted with about 1 to 3 substituents. When the number of substituents is 2 or more, they may be the same as or different from each other.

The “alkoxy” of the “optionally substituted alkoxy” includes C₁₋₆ alkoxy (e.g. methoxy, ethoxy, propoxy, isopropoxy, butoxy, isobutoxy, pentoxy, etc.). Examples of a “substituent” for the “optionally substituted alkoxy” are the same as those for the “optionally substituted alkyl” described above and the number of substituents used is also the same.

The “aryl” includes C₆₋₁₄ aryl (e.g. phenyl, 1-naphthyl, 2-naphthyl, biphenyl, 2-anthryl, etc.).

The “aryloxy” includes C₆₋₁₄ aryloxy (e.g. phenyloxy, 1-naphthyloxy, 2-naphthyloxy, etc.).

The “acyl” includes formyl, alkylcarbonyl, alkoxycarbonyl, carbamoyl, alkylcarbamoyl, alkylsulfinyl and alkylsulfonyl.

The “alkylcarbonyl” includes C₁₋₆ alkyl-carbonyl (e.g. acetyl, propionyl, etc.).

The “alkoxycarbonyl” includes C₁₋₆ alkoxy-carbonyl (e.g. methoxycarbonyl, ethoxycarbonyl, propoxycarbonyl, butoxycarbonyl, etc.).

The “alkylcarbamoyl” includes N—C₁₋₆ alkyl-carbamoyl (e.g. methylcarbamoyl, ethylcarbamoyl, etc.) and N,N-diC₁₋₆ alkyl-carbamoyl (e.g. N,N-dimethylcarbamoyl, N,N-diethylcarbamoyl, etc.).

The “alkylsulfinyl” includes C₁₋₇ alkylsulfinyl (e.g. methylsulfinyl, ethylsulfinyl, propylsulfinyl, isopropylsulfinyl, etc.).

The “alkylsulfonyl” includes C₁₋₇ alkylsulfonyl (e.g. methylsulfonyl, ethylsulfonyl, propylsulfonyl, isopropylsulfonyl, etc.).

The “acyloxy” includes alkylcarbonyloxy, alkoxycabonyloxy, carbamoyloxy, alkylcarbamoyloxy, alkylsulfinyloxy and alkylsulfonyloxy.

The “alkylcarbonyloxy” includes C₁₋₆ alkyl-carbonyloxy (e.g. acetyloxy, propionyloxy, etc.).

The “alkoxycarbonyloxy” includes C₁₋₆ alkoxy-carbonyloxy (e.g. methoxycarbonyloxy, ethoxycarbonyloxy, propoxycarbonyloxy, butoxycarbonyloxy, etc.).

The “alkylcarbamoyloxy” includes C₁₋₆ alkyl-carbamoyloxy (e.g. methylcarbamoyloxy, ethylcarbamoyloxy, etc.).

The “alkylsulfinyloxy” includes C₁₋₇ alkylsulfinyloxy (e.g. methylsulfinyloxy, ethylsulfinyloxy, propylsulfinyloxy, isopropylsulfinyloxy, etc.).

The “alkylsulfonyloxy” includes C₁₋₇ alkylsulfonyloxy (e.g. methylsulfonyloxy, ethylsulfonyloxy, propylsulfonyloxy, isopropylsulfonyloxy, etc.).

The “5- to 10-membered heterocyclic group” includes 5- to 10-membered (preferably 5- or 6-membered) heterocyclic groups containing 1 or more (e.g. 1 to 3) heteroatoms selected from a nitrogen atom, a sulfur atom and an oxygen atom in addition to carbon atoms, and specific examples thereof include 2- or 3-thienyl, 2-, 3- or 4-pyridyl, 2- or 3-furyl, 1-, 2- or 3-pyrrolyl, 2-, 3-, 4-, 5- or 8-quinolyl, 1-, 3-, 4- or 5-isoquinolyl, and 1-, 2- or 3-indolyl. Among them, preferred are 5- or 6-membered heterocyclic groups such as 1-, 2- or 3-pyrrolyl.

Ring A is preferably a benzene ring optionally substituted with 1 or 2 substituents selected from halogen, optionally halogenated C₁₋₄ alkyl, optionally halogenated C₁₋₄ alkoxy and 5- or 6-membered heterocyclic groups.

The “aralkyl” of the “optionally substituted aralkyl” represented by R¹ includes C₇₋₁₆ aralkyl (e.g. C₆₋₁₀ aryl C₁₋₆ alkyl such as benzyl and phenethyl). Examples of a “substituent” for the “optionally substituted aralkyl” are the same as those for the “optionally substituted alkyl” described above. The optionally substituted aralkyl may be substituted with about 1 to 4 substituents. When the number of substituents is 2 or more, they may be the same as or different from each other.

The “acyl” represented by R¹ includes the “acyl” mentioned above as a substituent for ring A.

The “acyloxy” represented by R¹ includes the “acyloxy” mentioned above as a substituent for ring A.

R¹ is preferably hydrogen.

The “optionally substituted alkyl” represented by R², R³ or R⁴ includes the “optionally substituted alkyl” mentioned above as a substituent for ring A.

The “optionally substituted alkoxy” represented by R², R³ or R⁴ includes the “optionally substituted alkoxy” mentioned above as a substituent for ring A.

The “optionally substituted amino” represented by R², R³ or R⁴ includes amino, mono-C₁₋₆ alkylamino (e.g. methylamino, ethylamino, etc.), mono-C₆₋₁₄ arylamino (e.g. phenylamino, 1-naphthylamino, 2-naphthylamino, etc.), di-C₁₋₆ alkylamino (e.g. dimethylamino, diethylamino, etc.) and di-C₆₋₁₄ arylamino (e.g. diphenylamino etc.).

R² is preferably C₁₋₆ alkyl, C₁₋₆ alkoxy, C₁₋₆ alkoxy-C₁₋₆ alkoxy or di-C₁₋₆ alkylamino. R² is more preferably C₁₋₃ alkyl or C₁₋₃ alkoxy.

R³ is preferably hydrogen, C₁₋₆ alkoxy-C₁₋₆ alkoxy or optionally halogenated C₁₋₆ alkoxy. R³ is more preferably C₁₋₃ alkoxy which may be halogenated or may be substituted with C₁₋₃ alkoxy.

R⁴ is preferably hydrogen or C₁₋₆ alkyl. R⁴ is more preferably hydrogen or C₁₋₃ alkyl (in particular, hydrogen).

Y is preferably nitrogen.

Specific examples of Compound (I) include the following compounds:
2-[[[3-methyl-4-(2,2,2-trifluoroethoxy)-2-pyridinyl]methyl]sulfinyl]-1H-benzimidazole,
2-[[(3,5-dimethyl-4-methoxy-2-pyridinyl)methyl]sulfinyl]-5-methoxy-1H-benzimidazole,
2-[[[4-(3-methoxypropoxy)-3-methyl-2-pyridinyl]methyl]sulfinyl]-1H-benzimidazole sodium salt,
5-difluoromethoxy-2-[[(3,4-dimethoxy-2-pyridinyl)methyl]sulfinyl]-1H-benzimidazole, 2-[(RS)-[(4-methoxy-3-methylpyridin-2-yl)methyl]sulfinyl]-5-(1H-pyrrol-1-yl)-1H-benzimidazole and the like.

Among these compounds, 2-[[[3-methyl-4-(2,2,2-trifluoroethoxy)-2-pyridinyl]methyl]sulfinyl]-1H-benzimidazole (lansoprazole) is preferable.

The aforementioned Compound (I) may be a racemate or an optically active isomer such as R-isomer or S-isomer. Preferred is an optically active isomer, for example, (R)-2-[[[3-methyl-4-(2,2,2-trifluoroethoxy)-2-pyridinyl]methyl]sulfinyl]-1H-benzimidazole (referred to as lansoprazole R-isomer in some cases).

A salt of Compound (I) is preferably a pharmaceutically acceptable salt and includes a salt with an inorganic base, a salt with an organic base and a salt with a basic amino acid.

Preferable examples of a salt with an inorganic base include alkali metal salts such as a sodium salt and a potassium salt; alkaline earth salts such as a calcium salt and a magnesium salt; and an ammonium salt.

Preferable examples of a salt with an organic base include salts with alkylamine (trimethylamine, triethylamine, etc.), heterocyclic amine (pyridine, picoline, etc.), alkanolamine (ethanolamine, diethanolamine, triethanolamine, etc.), dicyclohexylamine, N,N′-dibenzylethylenediamine or the like.

Preferable examples of a salt with a basic amino acid include salts with arginine, lysine, ornithine or the like.

Among these salts, preferred are alkali metal salts and alkaline earth metal salts. Inter alia, a sodium salt is preferable.

Prodrugs of Compound (I) and other benzimidazole or imidazopyridine PPI compounds are also relatively sensitive to acid and therefore they may be also used as an acid-labile active ingredient in the solid preparation of the present invention that has improved storage stability and can rapidly neutralize acid in stomach. Such prodrugs include prodrugs disclosed in WO 2003/27098, U.S. Pat. No. 4,045,563, U.S. Pat. No. 6,093,734, U.S. Pat. No. 5,039,806 and WO 2002/30920.

Compound (I) can be prepared by a per se known method, for example, a method described in JP-A 61-50978, U.S. Pat. No. 4,628,098, JP-A 10-195068 or WO 98/21201 or the similar method. An optically active isomer of Compound (I) can be obtained by an optical resolution method (e.g. fractional recrystallization method, chiral column method, diastereomer method, a method using microorganism or enzyme, etc.) or asymmetric oxidation. For example, the lansoprazole R isomer can be prepared by a method described in WO 00/78745, WO 01/83473, WO 01/87874 or WO 02/44167.

PPI used in the present invention is preferably a benzimidazole compound having anti-ulcer activity such as lansoprazole, omeprazole, rabeprazole, pantoprazole and ilaprazole; an imidazopyridine compound such as tenatoprazole; or an optically active isomer thereof or a pharmaceutically acceptable salt thereof.

The content of the benzimidazole compound in the solid preparation of the present invention varies depending on the kind of the active ingredient and a dose. For example, it is 0.0001 to 0.3 parts by weight, preferably 0.001 to 0.3 parts by weight, more preferably 0.002 to 0.2 parts by weight in 1 part by weight of the solid preparation of the present invention.

The alkaline earth metal carbonate used in the present invention includes magnesium carbonate and calcium carbonate for medical use.

The metal oxide and metal hydroxide used in the present invention are preferably in the form of a 1% (W/W) aqueous solution or 1% (W/W) aqueous suspension with pH 8.0 or higher. The metal oxide includes magnesium oxide, magnesium silicate (2MgO.3SiO₂.xH₂O), dry aluminum hydroxide gel (Al₂O₃.xH₂O) and magnesium aluminometasilicate (Al₂O₃.MgO.2SiO₂.xH₂O) for medical use. In particular, magnesium oxide is preferably used.

Preferred magnesium oxide is available for medicine, excellent in acid reactivity and has a neutralizing ability. Such magnesium oxide can be obtained by a conventional process or is commercially available and it is preferably so-called calcined magnesia, which is obtained by firing at a low temperature. Magnesium oxide fired at about 500° C. to about 1000° C. is generally preferable and, inter alia, magnesium oxide fired at about 600° C. to about 900° C., most preferably at about 800° C. is better from a viewpoint of a neutralizing ability. Among such magnesium oxide, preferred magnesium oxide can neutralize the environment in stomach after a preparation disintegrates in stomach and before an acid-labile active ingredient is released from the preparation, to increase the remaining rate of the active ingredient. Such preferred magnesium oxide generally has a BET specific surface area of 10 to 50 m²/g, preferably 20 to 50 m²/g.

Herein, the BET specific surface area is a specific surface area measured by a nitrogen gas adsorption method, which comprises measuring the amount of nitrogen gas adsorbed on the surface, including pores that nitrogen gas may enter, of a certain amount of magnesium oxide.

The magnesium oxide includes commercially available heavy magnesium oxide (manufactured by Kyowa Chemical Industry Co., Ltd.), heavy magnesium oxide (Tomita Pharmaceutical Co., Ltd.), heavy N magnesium oxide (manufactured by Kyowa Chemical Industry Co., Ltd.), and light magnesium oxide (manufactured by Kyowa Chemical Industry Co., Ltd.). Inter alia, heavy magnesium oxide (manufactured by Kyowa Chemical Industry Co., Ltd. or Tomita Pharmaceutical Co., Ltd.) and heavy N magnesium oxide (manufactured by Kyowa Chemical Industry Co., Ltd.) are preferable.

The metal hydroxide includes magnesium hydroxide, aluminum hydroxide, synthetic hydrotalcite (Mg₆Al₂(OH)₁₆CO₃.4H₂O), a coprecipitate of aluminum hydroxide and magnesium oxide, a coprecipitate of aluminum hydroxide, magnesium carbonate and calcium carbonate, and a coprecipitate of aluminum hydroxide and sodium hydrogen carbonate for medical use. Among them, magnesium hydroxide is preferable from a viewpoint of acid-reactivity, disintegration property and dissolution property of a preparation, and productivity. The alkaline earth metal carbonate, metal oxide and metal hydroxide may be used alone, or two or more of them may be used in combination. If necessary, a highly water-soluble basic additive may be further used jointly.

Some metal oxide or metal hydroxide may scrape the inner surface of manufacturing equipment in producing a preparation, so that the resulting tablet may have the wholly or partly black surface or have black dots or lines on the surface. In addition, some metal oxide or metal hydroxide may stick to punches of a tableting machine. Since the scrapability and punch-sticking property remarkably lower productivity of a preparation, when metal oxide or metal hydroxide having such properties is selected, it is used in combination with alkaline earth metal carbonate, metal oxide or metal hydroxide not having such properties. The combination can be further subjected to wet or dry granulation together with the following pharmaceutical additives (excipient, binder, disintegrating agent and the like) to suppress the scrapability and punch-sticking property.

In the case of a PPI preparation, single use of magnesium carbonate or calcium carbonate; two components-concomitant use of calcium carbonate and magnesium hydroxide or magnesium oxide; or three components-concomitant use of calcium carbonate, magnesium hydroxide and magnesium oxide is preferable from a viewpoint of compatibility with the active ingredient, dissolution property, disintegration property, productivity and the like.

The amount used of the alkaline earth metal carbonate and/or metal oxide and/or metal hydroxide is such an amount as to rapidly neutralize gastric acid in stomach, in order to prevent the substantial part of an active ingredient from being exposed to gastric acid and then destabilization of the active ingredient. Depending on the gastric acid neutralizing ability of individual alkaline earth metal carbonate, metal oxide or metal hydroxide, usually about 0.05 to 2000 parts by weight, preferably about 0.1 to 1000 parts by weight, more preferably about 0.1 to 800 parts by weight of the alkaline earth metal carbonate, metal oxide, metal hydroxide or a combination thereof (hereinafter, collectively referred to as the basic additive in some cases) is used per 1 part by weight of an acid-labile active ingredient. For example, 0.1 to 1500 parts by weight, preferably 0.5 to 800 parts by weight, more preferably 0.1 to 400 parts by weight of the basic additive is used per 1 part by weight of a benzimidazole compound. When the active ingredient is a benzimidazole compound, the intragastric pH usually rises after administration. The basic additive is preferably used in such an amount as to raise the intragastric pH from the normal pH to pH 3 or higher within about 60 minutes, more preferably within about 40 minutes after administration.

The solid preparation of the present invention may contain at least one highly water-soluble basic additive, if necessary, in addition to the alkaline earth metal carbonate and/or metal oxide and/or metal hydroxide. The highly water-soluble basic additive includes pharmaceutical additives having antacid activity such as trometamol, disodium succinate, sodium hydrogen phosphate, trisodium phosphate, dipotassium phosphate, L-arginine and meglumine. These may be used alone or two or more of these may be used in combination.

The highly water-soluble basic additive is also used in such an amount as to rapidly neutralize gastric acid in stomach, in order to prevent the substantial part of an active ingredient from being exposed to gastric acid and then destabilization of the active ingredient. Depending on the gastric acid neutralizing ability of individual highly water-soluble basic additive, the total amount used of the metal oxide, the metal hydroxide and the highly water-soluble basic additive is about 0.05 to 2000 parts by weight, preferably about 0.1 to 1200 parts by weight, more preferably about 0.1 to 800 parts by weight per 1 part by weight of an acid-labile active ingredient. When the active ingredient is a benzimidazole compound, the total amount used of the metal oxide, the metal hydroxide and the highly water-soluble basic additive is usually 0.1 to 1800 parts by weight, preferably 0.5 to 1000 parts by weight, more preferably 1 to 800 parts by weight per 1 part by weight of a benzimidazole compound. The highly water-soluble basic additive, in combination with the metal oxide and the metal hydroxide, is preferably used in such an amount as to raise the intragastric pH from the normal pH to pH 3 or higher within about 60 minutes, more preferably within about 40 minutes after administration.

The chewable tablet of the present invention may contain further additives for tablet forming, such as an excipient (e.g. glucose, fructose, lactose, sucrose, D-mannitol, erythritol, maltitol, trehalose, sorbitol, corn starch, potato starch, wheat starch, rice starch, microcrystalline cellulose, silicic anhydride, anhydrous calcium phosphate, precipitated calcium carbonate, calcium silicate, etc.), a binder (e.g. hydroxypropylcellulose, hydroxypropylmethylcellulose, polyvinylpyrrolidone, methylcellulose, polyvinyl alcohol, sodium carboxymethylcellulose, partially pregelatinized starch, pregelatinized starch, sodium alginate, pullulan, gum arabic powder, gelatin, etc.), a disintegrant (e.g. low-substituted hydroxypropylcellulose, carmellose, carmellose calcium, carboxymethyl starch sodium, croscarmellose sodium, crospovidone, hydroxypropylstarch, etc.), a corrigent (e.g. citric acid, ascorbic acid, tartaric acid, malic acid, dipotassium glycyrrhizinate, sodium glutamate, sodium 5′-inosinate, sodium 5′-guanylate, etc.), a sweetener (e.g. aspartame, acesulfame potassium, sormatin, saccharin sodium, dipotassium glycyrrhizinate, etc.), a surfactant (e.g. Polysorbate, polyoxyethylene.polyoxypropylene copolymer, sodium lauryl sulfate), a flavor (e.g. lemon oil, orange oil, menthol, peppermint oil, etc.), a lubricant (e.g. magnesium stearate, sucrose fatty acid ester, sodium stearyl fumarate, stearic acid, talc, polyethylene glycol, etc.), a coloring agent (e.g. Food yellow No. 5, Food blue No. 2, ferric oxide, yellow ferric oxide, etc.), and an antioxidant (e.g. sodium ascorbate, L-cysteine, sodium sulfite, etc.).

The particle diameter of raw material used in the present invention is not particularly limited, but it is preferably 500 μm or smaller (preferably 300 μm or smaller) from a viewpoint of productivity.

For the purpose of further stabilizing an acid-labile active ingredient, the chewable tablet of the present invention may be a chewable tablet comprising a group containing an acid-labile active ingredient and at least one basic substance selected from alkaline earth metal carbonate, metal oxide and metal hydroxide, and another group not containing an acid-labile active ingredient and containing at least one ingredient selected from alkaline earth metal carbonate, metal oxide and metal hydroxide. More specifically, an acid-labile active ingredient and at least one basic substance selected from alkaline earth metal carbonate, metal oxide and metal hydroxide are granulated, if necessary, together with other excipients and the like; and at least one ingredient selected from alkaline earth metal carbonate, metal oxide and metal hydroxide is granulated together with other excipients and the like and without an acid-labile active ingredient. Then, these two groups of granules may be mixed and formulated into a preparation by a conventional method. Alternatively, a layer containing an acid-labile active ingredient and at least one basic substance selected from alkaline earth metal carbonate, metal oxide and metal hydroxide and if necessary, other excipients and the like, and another layer not containing an active ingredient and only containing at least one basic substance selected from alkaline earth metal carbonate, metal oxide and metal hydroxide and if necessary, other excipients and the like may be stacked to obtain a preparation (e.g. layered tablet such as two-layered tablet, or a preparation comprising the core layer containing an active ingredient and the coating layer not containing an active ingredient, etc.).

A more preferable aspect of the solid preparation of the present invention is a chewable tablet comprising a group which contains an acid-labile active ingredient and alkaline earth metal carbonate (preferably calcium carbonate), and a group which does not contain an acid-labile active ingredient and contains at least one ingredient selected from metal oxide and metal hydroxide.

Since alkaline earth metal carbonate such as calcium carbonate is highly effective in improving the stability of an acid-labile active ingredient, it is preferable that alkaline earth metal carbonate is located near an acid-labile active ingredient.

A preferred combination of metal oxide and metal hydroxide is magnesium oxide and magnesium hydroxide. As described above, although metal oxide such as magnesium oxide is effective in rapidly neutralizing gastric acid, it is preferably used in combination with metal hydroxide such as magnesium hydroxide in order to improve productivity. The weight ratio between metal oxide such as magnesium oxide and metal hydroxide such as magnesium hydroxide is preferably 1:0.2 to 1:50.

A more preferable aspect of the present invention is a chewable tablet comprising a group which contains about 0.001 to about 0.3 parts by weight of lansoprazole or an optically active isomer thereof or a salt thereof per 1 part by weight of the tablet and about 0.2 to about 200 parts by weight of calcium carbonate per 1 part by weight of lansoprazole or an optically active isomer thereof or a salt thereof, and a group which does not contain lansoprazole or an optically active isomer thereof or a salt thereof and contains total about 0.2 to about 200 parts by weight of magnesium oxide and magnesium hydroxide per 1 part by weight of lansoprazole or an optically active isomer thereof or a salt thereof.

A process for preparing the solid preparation which is a chewable tablet of the present invention may be a per se known method. For example, a benzimidazole compound, alkaline earth metal carbonate and/or metal oxide and/or metal hydroxide, a highly water-soluble basic additive having antacid activity if necessary, an excipient, a binder, a disintegrant, a lubricant, a corrigent, a sweetener, a coloring agent, a flavor and the like are combined in their appropriate amounts and then directly compressed into a chewable tablet. Alternatively, preferably, a granule may be obtained by a wet granulation method and the resulting granule may be formulated into a chewable tablet.

Herein, the wet granulation method comprises granulating a mixture of a benzimidazole compound, alkaline earth metal carbonate and/or metal oxide and/or metal hydroxide, an excipient and the like using a solution or suspension of a binder and then drying to obtain granulated powder. The granulation step may be performed by any suitable method such as extrusion, fluidization, rolling, centrifugation, stirring or spraying. This granulated powder is mixed with appropriate amounts of a disintegrant, a lubricant, a corrigent, a sweetener, a flavor, a coloring agent and the like and then compressed into a chewable tablet.

Alternatively, for example, a mixture of a benzimidazole compound, alkaline earth metal carbonate and/or metal oxide and/or metal hydroxide, an excipient and the like is granulated using a solution or suspension of a binder to obtain a group containing an active ingredient; and a mixture of alkaline earth metal carbonate and/or metal oxide and/or metal hydroxide and/or a highly water-soluble basic additive, an excipient and the like is granulated using a solution or suspension of a binder to obtain another group not containing an active ingredient. Then, these two groups are combined together with appropriate amounts of a disintegrant, a lubricant, a corrigent, a sweetener, a flavor, a coloring agent and the like and compressed into a chewable tablet. Further alternatively, for example, a layer comprising the part containing a benzimidazole compound in combination with appropriate amounts of a disintegrant, a lubricant, a corrigent, a sweetener, a flavor, a coloring agent and the like, and another layer comprising the part not containing an active ingredient in combination with appropriate amounts of a disintegrant, a lubricant, a corrigent, a sweetener, a flavor, a coloring agent and the like are compressed into a layered chewable tablet.

In producing the preparation of the present invention wherein its ingredients are divided into two groups, an additive having a binding ability (e.g. hydroxypropylcellulose, hydroxypropylmethylcellulose, hydroxyethylcellulose, polyvinylpyrrolidone, methylcellulose, polyvinyl alcohol, carboxymethylcellulose sodium, partially pregelatinized starch, pregelatinized starch, sodium alginate, plullan, gum arabic powder, gelatin, polyethylene oxide, carboxymethylethylcellulose, carboxyvinyl polymer, ethylcellulose, ethyl acrylate.methyl methacrylate.trimethylammoniumethyl methacrylate copolymer, etc.) may be incorporated in a part containing an active ingredient to delay dissolution of the active ingredient. A part containing an active ingredient may be also coated with an ingredient comprising hydroxypropylmethylcellulose, hydroxypropylcellulose, polyvinyl alcohol, polyvinylpyrrolidone, ethylcellulose, ethyl acrylate methyl methacrylate.trimethylammoniumethyl methacrylate copolymer, methacrylic acid polymer, hydroxypropylmethylcellulose phthalate, hydroxypropylmethylcellulose acetate succinate, cellulose acetate succinate or the like to delay dissolution of the active ingredient.

The chewable tablet of the present invention may have such hardness that one can chew up it. Preferably, the hardness is 30N to 5ON, more preferably 50N to 300N.

The hardness can be measured using, for example, a Toyama-type tablet hardness tester (Toyama Sangyo Co., Ltd., Pharma Test WHT-1 type (PHARMA TEST APPARATBBAU GMBH)).

Although the chewable tablet of the present invention may have big size because it is taken by chewing up, a chewable tablet with a diameter of 25 mm or less is easy to take and preferred. The chewable tablet of the present invention may be in round, oval, oblong or triangle form or the like.

The solid preparation of the present invention can be orally administered by chewing up or disintegrated with saliva in an oral cavity without chewing up and then swallowed.

When the solid preparation of the present invention comprises a benzimidazole compound represented by the formula (I) such as lansoprazole or an optically active isomer thereof as an active ingredient, it is useful as a medicament because the compound has excellent anti-ulcer activity, gastric acid secretion inhibitory activity, mucosa protecting activity, anti-helicobacter pylori activity and the like and has low toxicity. In this case, the solid preparation of the present invention can be orally administered to a mammal (e.g. human, monkey, sheep, horse, dog, cat, rabbit, rat, mouse, etc.) for the purpose of treating or preventing peptic ulcer (e.g. gastric ulcer, duodenal ulcer, stomal ulcer, Zollinger-Ellison syndrome, etc.), gastritis, GERD (Gastroesophageal Reflux Diseases; reflux esophagitis, symptomatic GERD, etc.), NUD (Non Ulcer Dyspepsia), stomach cancer (including stomach cancer associated with promotion of interleukin-1β production due to interleukin-1 gene polymorphism), gastric MALT lymphoma and the like, eliminating Helicobacter pylori, suppressing upper digestive tract bleeding due to peptic ulcer, acute stress ulcer or hemorrhagic gastritis, suppressing upper digestive tract bleeding due to invasive stress (stress caused by big operation requiring intensive management after operation or by cerebrovascular disorder, head trauma, multi-organ failure or diffuse burn requiring intensive care), treating or preventing ulcer caused by a non-steroidal anti-inflammatory agent; or treating or preventing gastric hyperacidity or ulcer due to post-operation stress. In order to eliminate Helicobacter pylori, it is preferable to use the solid preparation of the present invention in combination with a penicillin antibiotic (e.g. amoxicillin, etc.) and an erythromycin antibiotic (e.g. clarithromycin, etc.).

Inter alia, the preparation of the present invention is suitably used as an agent for treating or preventing GERD (Symptomatic GERD, reflux esophagitis, etc.). The chewable tablet of the present invention is also suitable to children or the elderly as long as they can chew the tablet.

The daily dose of the solid preparation of the present invention varies depending on the severity of a symptom, the age, sex and weight of a subject to be administered, a timing and interval of administration, the kind of an active ingredient and the like and is not particularly limited. For example, when the solid preparation of the present invention is orally administered to an adult (60 kg) as an anti-ulcer agent, the daily dose is about 0.5 to 1500 mg/day, preferably about 5 to 150 mg/day of the active ingredient. The benzimidazole compound-containing preparation of the present invention may be administered once a day or 2 to 3 divided doses per day.

The present invention will be explained in more detail below by way of Examples and Experimental Examples, but the present invention is not limited to them.

### EXAMPLE 1

Production of a Group Containing an Active Ingredient

A fluid bed granulator was charged with 150 g of lansoprazole, 1875 g of calcium carbonate and 635 g of D-mannitol. A solution of 120 g of hydroxypropylcellulose in 1880 g of purified water was sprayed into the granulator, and the mixture was granulated and dried to obtain 2695 g of a granulated powder.

Production of a Group not Containing an Active Ingredient

A fluid bed granulator was charged with 1450 g of magnesium hydroxide, 853.2 g of D-mannitol, 96 g of aspartame and 64.8 g of crospovidone. A solution of 96 g of hydroxypropylcellulose in 1504 g of purified water was sprayed into the granulator, and the mixture was granulated and dried to obtain 2503 g of a granulated powder.

Into a bag, 1668 g of the group containing an active ingredient, 1920 g of the group not containing an active ingredient, 360 g of crystalline cellulose (Ceolas KG-801), 180 g of crospovidone and 72 g of magnesium stearate were put and mixed to obtain a mixed powder. The mixed powder was compressed into tablets (1400 mg/one tablet) using a rotary tableting machine with a punch (17 mmφ flat-faced with beveled edge). Blackening was not observed in the resulting tablets.

### EXAMPLE 2

Production of a Group Containing an Active Ingredient

A fluid bed granulator was charged with 30 g of lansoprazole, 750 g of calcium carbonate and 284 g of D-mannitol. A solution of 48 g of hydroxypropylcellulose in 752 g of purified water was sprayed into the granulator, and the mixture was granulated and dried to obtain 1066 g of a granulated powder.

Production of a Group not Containing an Active Ingredient

A fluid bed granulator was charged with 725 g of magnesium hydroxide, 426.6 g of D-mannitol, 48 g of aspartame and 32.4 of crospovidone. A solution of 48 g of hydroxypropylcellulose in 752 g of purified water was sprayed into the granulator, and the mixture was granulated and dried to obtain 1161 g of a granulated powder.

Into a bag, 834 g of the group containing an active ingredient, 960 g of the group not containing an active ingredient, 180 g of crystalline cellulose (Ceolas KG-801), 90 g of crospovidone and 36 g of magnesium stearate were put and mixed to obtain a mixed powder. The mixed powder was compressed into tablets (1400 mg/one tablet) using a rotary tableting machine with a punch (17 mmφ flat-faced with beveled edge). Blackening was not observed in the resulting tablets.

### EXAMPLE 3

Production of a Group Containing an Active Ingredient

A fluid bed granulator was charged with 60 g of lansoprazole, 750 g of calcium carbonate and 254 g of D-mannitol. A solution of 48 g of hydroxypropylcellulose in 752 g of purified water was sprayed into the granulator, and the mixture was granulated and dried to obtain 1070.5 g of a granulated powder.

Production of a Group not Containing an Active Ingredient

A fluid bed granulator was charged with 435 g of magnesium hydroxide, 716.6 g of D-mannitol, 48 g of aspartame and 32.4 g of crospovidone. A solution of 48 g of hydroxypropylcellulose in 752 g of purified water was sprayed into the granulator, and the mixture was granulated and dried to obtain 1245.4 g of a granulated powder.

Into a bag, 834 g of the group containing an active ingredient, 960 g of the group not containing an active ingredient, 180 g of crystalline cellulose (Ceolas KG-801), 90 g of crospovidone and 36 g of magnesium stearate were put and mixed to obtain a mixed powder. The mixed powder was compressed into tablets (1400 mg/one tablet) using a rotary tableting machine with a punch (17 mmφ flat-faced with beveled edge). Blackening was not observed in the resulting tablets.

### EXAMPLE 4

Production of a Group Containing an Active Ingredient

A fluid bed granulator was charged with 75 g of lansoprazole, 937.5 g of calcium carbonate and 317.5 g of D-mannitol. A solution of 60 g of hydroxypropylcellulose in 940 g of purified water was sprayed into the granulator, and the mixture was granulated and dried to obtain 1306 g of a granulated powder.

Production of a Group not Containing an Active Ingredient

A fluid bed granulator was charged with 300 g of magnesium oxide, 651.6 g of D-mannitol, 48 g of aspartame and 32.4 g of crospovidone. A solution of 48 g of hydroxypropylcellulose in 752 g of purified water was sprayed into the granulator, and the mixture was granulated and dried to obtain 1053 g of a granulated powder.

Into a bag, 333.6 g of the group containing an active ingredient, 324 g of the group not containing an active ingredient, 72 g of crystalline cellulose (Ceolas KG-801), 36 g of crospovidone and 14.4 g of magnesium stearate were put and mixed to obtain a mixed powder. The mixed powder was compressed into tablets (1300 mg/one tablet) using a rotary tableting machine with a punch (16 mmφ flat-faced with beveled edge). Blackening was not observed in the resulting tablets.

### EXAMPLE 5

Production of a Group Containing an Active Ingredient

A fluid bed granulator was charged with 75 g of lansoprazole, 937.5 g of calcium carbonate and 317.5 g of D-mannitol. A solution of 60 g of hydroxypropylcellulose in 940 g of purified water was sprayed into the granulator, and the mixture was granulated and dried to obtain 1306 g of a granulated powder.

Production of a Group not Containing an Active Ingredient

A fluid bed granulator was charged with 400 g of magnesium oxide, 145 g of magnesium hydroxide, 406.6 g of D-mannitol, 48 g of aspartame and 32.4 g of crospovidone. A solution of 48 g of hydroxypropylcellulose in 752 g of purified water was sprayed into the granulator, and the mixture was granulated and dried to obtain 1027 g of a granulated powder.

Into a bag, 333.6 g of the group containing an active ingredient, 324 g of the group not containing an active ingredient, 72 g of crystalline cellulose (Ceolas KG-801), 36 g of crospovidone and 14.4 g of magnesium stearate were put and mixed to obtain a mixed powder. The mixed powder was compressed into tablets (1300 mg/one tablet) using a rotary tableting machine with a punch (16 mmφ flat-faced with beveled edge). Blackening was not observed in the resulting tablets.

### EXPERIMENTAL EXAMPLE

The tablets obtained in Examples 1, 2, 3, 4 and 5 were placed in a glass bottle and kept at 60° C. for 2 weeks. The tablets after storage were measured for the remaining rate of lansoprazole by liquid chromatography.

| | Remaining rate (%) of lansoprazole |
|---|---|
| Example 1 | 102 |
| Example 2 | 96 |
| Example 3 | 98 |
| Example 4 | 100 |
| Example 5 | 97 |

### EXAMPLE 6

Production of a Group Containing an Active Ingredient

A fluid bed granulator was charged with 420 g of lansoprazole, 7000 g of calcium carbonate and 6543.6 g of D-mannitol. A solution of 582.4 g of hydroxypropylcellulose in 8124.3 g of purified water and a dispersion of 14 g of ferric oxide in 1000 g of water were mixed and then sprayed into the granulator. The mixture was granulated and dried to obtain 13989.0 g of a granulated powder.

Production of a Group not Containing an Active Ingredient

A fluid bed granulator was charged with 2100 g of magnesium oxide, 6090 g of magnesium hydroxide, 406.6 g of D-mannitol, 945 g of aspartame and 588 g of crospovidone. A solution of 588 g of hydroxypropylcellulose in 8212 g of purified water and a dispersion of 14.7 g of ferric oxide in 1000 g of water were mixed and then sprayed into the granulator. The mixture was granulated and dried to obtain 13945.0 g of a granulated powder.

3640 g of the group containing an active ingredient, 4760 g of the group not containing an active ingredient, 805 g of crystalline cellulose (Ceolas KG-802), 350 g of crospovidone, 98 g of strawberry flavor and 147 g of magnesium stearate were mixed to obtain a mixed powder. The mixed powder was compressed into tablets (1400 mg/one tablet) using a rotary tableting machine with a punch (16 mmφ flat-faced with beveled edge). Blackening was not observed in the resulting tablets.

### EXAMPLE 7

Production of a Group Containing an Active Ingredient

A fluid bed granulator was charged with 840 g of lansoprazole, 7000 g of calcium carbonate and 6123.6 g of D-mannitol. A solution of 582.4 g of hydroxypropylcellulose in 8124.3 g of purified water and a dispersion of 14 g of ferric oxide in 1000 g of water were mixed and then sprayed into the granulator. The mixture was granulated and dried to obtain 13865.8 g of a granulated powder.

Production of a Group not Containing an Active Ingredient

A fluid bed granulator was charged with 2100 g of magnesium oxide, 6090 g of magnesium hydroxide, 406.6 g of D-mannitol, 945 g of aspartame and 588 g of crospovidone. A solution of 588 g of hydroxypropylcellulose in 8212 g of purified water and a dispersion of 14.7 g of ferric oxide in 1000 g of water were mixed and then sprayed into the granulator. The mixture was granulated and dried to obtain 13945.0 g of a granulated powder.

3640 g of the group containing an active ingredient, 4760 g of the group not containing an active ingredient, 805 g of crystalline cellulose (Ceolas KG-802), 350 g of crospovidone, 98 g of strawberry flavor and 147 g of magnesium stearate were mixed to obtain a mixed powder. The mixed powder was compressed into tablets (1400 mg/one tablet) using a rotary tableting machine with a punch (16 mmφ flat-faced with beveled edge). Blackening was not observed in the resulting tablets.

### EXAMPLE 8

Production of a Group Containing an Active Ingredient

A fluid bed granulator was charged with 405 g of lansoprazole, 6750 g of calcium carbonate and 7192.8 g of D-mannitol. A solution of 648 g of hydroxypropylcellulose in 9152 g of purified water and a dispersion of 16.2 g of ferric oxide in 1000 g of water were mixed and then sprayed into the granulator. The mixture was granulated and dried to obtain 14662.2 g of a granulated powder.

Production of a Group not Containing an Active Ingredient

A fluid bed granulator was charged with 1500 g of magnesium oxide, 6525 g of magnesium hydroxide, 4354.5 g of D-mannitol, 900 g of aspartame and 315 g of crospovidone. A solution of 493.5 g of hydroxypropylcellulose in 6731.5 g of purified water and a dispersion of 12 g of ferric oxide in 1000 g of water were mixed and then sprayed into the granulator. The mixture was granulated and dried to obtain 13780.1 g of a granulated powder.

3892 g of the group containing an active ingredient, 6580 g of the group not containing an active ingredient, 721 g of crystalline cellulose (Ceolas KG-802), 420 g of crospovidone, 119 g of strawberry flavor and 168 g of magnesium stearate were mixed to obtain a mixed powder. The mixed powder was compressed into tablets (1700 mg/one tablet) using a rotary tableting machine with a punch (18 mmφ flat-faced with beveled edge). Blackening was not observed in the resulting tablets.

### EXAMPLE 9

Production of a Group Containing an Active Ingredient

A fluid bed granulator was charged with 810 g of lansoprazole, 6750 g of calcium carbonate and 6787.8 g of D-mannitol. A solution of 648 g of hydroxypropylcellulose in 9152 g of purified water and a dispersion of 16.2 g of ferric oxide in 1000 g of water were mixed and then sprayed into the granulator. The mixture was granulated and dried to obtain 13967.9 g of a granulated powder.

Production of a Group not Containing an Active Ingredient

A fluid bed granulator was charged with 1500 g of magnesium oxide, 6525 g of magnesium hydroxide, 4354.5 g of D-mannitol, 900 g of aspartame and 315 g of crospovidone. A solution of 493.5 g of hydroxypropylcellulose in 6731.5 g of purified water and a dispersion of 12 g of ferric oxide in 1000 g of water were mixed and then sprayed into the granulator. The mixture was granulated and dried to obtain 13780.1 g of a granulated powder.

3892 g of the group containing an active ingredient, 6580 g of the group not containing an active ingredient, 721 g of crystalline cellulose (Ceolas KG-802), 420 g of crospovidone, 119 g of strawberry flavor and 168 g of magnesium stearate were mixed to obtain a mixed powder. The mixed powder was compressed into tablets (1700 mg/one tablet) using a rotary tableting machine with a punch (18 mmφ flat-faced with beveled edge). Blackening was not observed in the resulting tablets.

### EXAMPLE 10

Production of a Group Containing an Active Ingredient

A fluid bed granulator is charged with 75 g of lansoprazole, 1250 g of calcium carbonate and 1168.5 g of D-mannitol. Into the granulator 1735.8 g of a 6% aqueous solution of hydroxypropylcellulose containing 2.5 g of ferric oxide is sprayed and the mixture is granulated.

Production of a Group not Containing an Active Ingredient

A fluid bed granulator is charged with 800 g of magnesium oxide, 580 g of magnesium hydroxide, 731.2 g of D-mannitol, 208 g of aspartame and 70 g of crospovidone. Into the granulator 1469.5 g of a 6% aqueous solution of hydroxypropylcellulose containing 2.8 g of ferric oxide is sprayed and the mixture is granulated.

1560 g of the group containing an active ingredient, 1860 g of the group not containing an active ingredient, 378 g of crystalline cellulose (Ceolas KG-802), 150 g of crospovidone, 40.5 g of strawberry flavor and 61.5 g of magnesium stearate are mixed to obtain a mixed powder. The mixed powder is compressed into tablets (1350 mg/one tablet) using a rotary tableting machine with a punch (16 mmφ flat-faced with beveled edge).

### EXAMPLE 11

### Production of a Group Containing an Active Ingredient

A fluid bed granulator is charged with 150 g of lansoprazole, 1250 g of calcium carbonate and 1093.5 g of D-mannitol. Into the granulator 1735.8 g of a 6% aqueous solution of hydroxypropylcellulose containing 2.5 g of ferric oxide is sprayed and the mixture is granulated.

Production of a Group not Containing an Active Ingredient

A fluid bed granulator is charged with 800 g of magnesium oxide, 580 g of magnesium hydroxide, 731.2 g of D-mannitol, 208 g of aspartame and 70 g of crospovidone. Into the granulator 1469.5 g of a 6% aqueous solution of hydroxypropylcellulose containing 2.8 g of ferric oxide is sprayed and the mixture is granulated.

1560 g of the group containing an active ingredient, 1860 g of the group not containing an active ingredient, 378 g of crystalline cellulose (Ceolas KG-802), 150 g of crospovidone, 40.5 g of strawberry flavor and 61.5 g of magnesium stearate are mixed to obtain a mixed powder. The mixed powder is compressed into tablets (1350 mg/one tablet) using a rotary tableting machine with a punch (16 mmφ flat-faced with beveled edge).

### EXAMPLE 12

Production of a Group Containing an Active Ingredient

A fluid bed granulator is charged with 75 g of lansoprazole, 1250 g of calcium carbonate and 1168.5 g of D-mannitol. Into the granulator 1735.8 g of a 6% aqueous solution of hydroxypropylcellulose containing 2.5 g of ferric oxide is sprayed and the mixture is granulated.

Production of a Group not Containing an Active Ingredient

A fluid bed granulator is charged with 700 g of magnesium oxide, 1015 g of magnesium hydroxide, 732.2 g of D-mannitol, 217 g of aspartame and 52.5 g of crospovidone. Into the granulator 1636.1 g of a 6% aqueous solution of hydroxypropylcellulose containing 2.8 g of ferric oxide is sprayed and the mixture is granulated.

1560 g of the group containing an active ingredient, 2415 g of the group not containing an active ingredient, 409.2 g of crystalline cellulose (Ceolas KG-802), 180 g of crospovidone, 46.8 g of strawberry flavor and 69 g of magnesium stearate are mixed to obtain a mixed powder. The mixed powder is compressed into tablets (1560 mg/one tablet) using a rotary tableting machine with a punch (18 mmφ flat-faced with beveled edge).

### EXAMPLE 13

Production of a Group Containing an Active Ingredient

A fluid bed granulator is charged with 150 g of lansoprazole, 1250 g of calcium carbonate and 1093.5 g of D-mannitol. Into the granulator 1735.8 g of a 6% aqueous solution of hydroxypropylcellulose containing 2.5 g of ferric oxide is sprayed and the mixture is granulated.

Production of a Group not Containing an Active Ingredient

A fluid bed granulator is charged with 700 g of magnesium oxide, 1015 g of magnesium hydroxide, 732.2 g of D-mannitol, 217 g of aspartame and 52.5 g of crospovidone. Into the granulator 1636.1 g of a 6% aqueous solution of hydroxypropylcellulose containing 2.8 g of ferric oxide is sprayed and the mixture is granulated.

1560 g of the group containing an active ingredient, 2415 g of the group not containing an active ingredient, 409.2 g of crystalline cellulose (Ceolas KG-802), 180 g of crospovidone, 46.8 g of strawberry flavor and 69 g of magnesium stearate are mixed to obtain a mixed powder. The mixed powder is compressed into tablets (1560 mg/one tablet) using a rotary tableting machine with a punch (18 mmφ flat-faced with beveled edge).

### EXAMPLE 14

Production of a Group Containing an Active Ingredient

A fluid bed granulator is charged with 75 g of lansoprazole, 1250 g of calcium carbonate and 1168.5 g of D-mannitol. Into the granulator 1735.8 g of a 6% aqueous solution of hydroxypropylcellulose containing 2.5 g of ferric oxide is sprayed and the mixture is granulated.

Production of a Group not Containing an Active Ingredient

A fluid bed granulator is charged with 1050 g of magnesium oxide, 507.5 g of magnesium hydroxide, 697.2 g of D-mannitol, 210 g of aspartame and 52.5 g of crospovidone. Into the granulator 1519.5 g of a 6% aqueous solution of hydroxypropylcellulose containing 2.8 g of ferric oxide is sprayed and the mixture is granulated.

1560 g of the group containing an active ingredient, 2238 g of the group not containing an active ingredient, 409.5 g of crystalline cellulose (Ceolas KG-802), 180 g of crospovidone, 45 g of strawberry flavor and 67.5 g of magnesium stearate are mixed to obtain a mixed powder. The mixed powder is compressed into tablets (1500 mg/one tablet) using a rotary tableting machine with a punch (18 mmφ flat-faced with beveled edge).

### EXAMPLE 15

Production of a Group Containing an Active Ingredient

A fluid bed granulator is charged with 150 g of lansoprazole, 1250 g of calcium carbonate and 1093.5 g of D-mannitol. Into the granulator 1735.8 g of a 6% aqueous solution of hydroxypropylcellulose containing 2.5 g of ferric oxide is sprayed and the mixture is granulated.

Production of a Group not Containing an Active Ingredient

A fluid bed granulator is charged with 1050 g of magnesium oxide, 507.5 g of magnesium hydroxide, 697.2 g of D-mannitol, 210 g of aspartame and 52.5 g of crospovidone. Into the granulator 1519.5 g of a 6% aqueous solution of hydroxypropylcellulose containing 2.8 g of ferric oxide is sprayed and the mixture is granulated.

1560 g of the group containing an active ingredient, 2238 g of the group not containing an active ingredient, 409.5 g of crystalline cellulose (Ceolas KG-802), 180 g of crospovidone, 45 g of strawberry flavor and 67.5 g of magnesium stearate are mixed to obtain a mixed powder. The mixed powder is compressed into tablets (1500 mg/one tablet) using a rotary tableting machine with a punch (18 mmφ flat-faced with beveled edge).

1. A chewable tablet, comprising a group which contains an acid-labile active ingredient and at least one basic substance selected from alkaline earth metal carbonate, metal oxide and metal hydroxide, and a group which does not contain an acid-labile active ingredient and contains at least one ingredient selected from alkaline earth metal carbonate, metal oxide and metal hydroxide.

2. The chewable tablet according to claim 1, which is not enteric-coated.

3. The chewable tablet according to claim 1, wherein the acid-labile active ingredient is a proton pump inhibitor (PPI).

4. The chewable tablet according to claim 3, wherein the PPI is a benzimidazole compound.

5. The chewable tablet according to claim 4, wherein the benzimidazole compound is a compound represented by the formula (I): 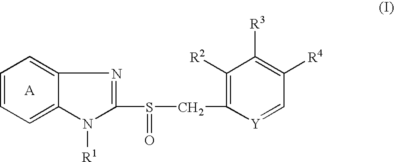
wherein ring A represents an optionally substituted benzene ring, R¹ represents hydrogen, optionally substituted aralkyl, acyl or acyloxy, R², R³ and R⁴ may be the same or different and each represents hydrogen, optionally substituted alkyl, optionally substituted alkoxy or optionally substituted amino, and Y represents nitrogen or CH, or an optically active isomer thereof or a salt thereof.

6. The chewable tablet according to claim 4, wherein the benzimidazole compound is lansoprazole, omeprazole, rabeprazole, pantoprazole, ilaprazole or an optically active isomer thereof or a salt thereof.

7. The chewable tablet according to claim 3, wherein the PPI is an imidazopyridine compound.

8. The chewable tablet according to claim 7, wherein the imidazopyridine compound is tenatoprazole or an optically active isomer thereof or a salt thereof.

9. The chewable tablet according to claim 1, wherein the alkaline earth metal carbonate is calcium carbonate or magnesium carbonate.

10. The chewable tablet according to claim 1, wherein the metal oxide and the metal hydroxide are in the form of a 1% (W/W) aqueous solution or suspension with pH 8.0 or higher.

11. The chewable tablet according to claim 1, containing at least one metal oxide selected from the group consisting of magnesium oxide, magnesium silicate, dry aluminum hydroxide gel and magnesium aluminometasilicate.

12. The chewable tablet according to claim 1, containing at least one metal hydroxide selected from the group consisting of magnesium hydroxide, aluminum hydroxide, synthetic hydrotalcite, a coprecipitate of aluminum hydroxide and magnesium hydroxide, a coprecipitate of aluminum hydroxide, magnesium carbonate and calcium carbonate, and a coprecipitate of aluminum hydroxide and sodium hydrogen carbonate.

13. The chewable tablet according to claim 1, wherein the alkaline earth metal carbonate is calcium carbonate.

14. The chewable tablet according to claim 1, wherein the metal oxide is magnesium oxide.

15. The chewable tablet according to claim 1, wherein the metal hydroxide is magnesium hydroxide.

16. The chewable tablet according to claim 4, containing 0.1 to 1500 parts by weight of at least one ingredient selected from alkaline earth metal carbonate, metal oxide and metal hydroxide per 1 part by weight of the benzimidazole compound.

17. A chewable tablet, comprising a group which contains an acid-labile active ingredient and alkaline earth metal carbonate, and a group which does not contain an acid-labile active ingredient and contains at least one ingredient selected from metal oxide and metal hydroxide.

18. The chewable tablet according to claim 17, wherein the alkaline earth metal carbonate is calcium carbonate, the metal oxide is magnesium oxide and the metal hydroxide is magnesium hydroxide.

19. A chewable tablet, comprising a group which contains 0.001 to 0.3 parts by weight of lansoprazole or an optically active isomer thereof or a salt thereof per 1 part by weight of said tablet and 0.2 to 200 parts by weight of calcium carbonate per 1 part by weight of lansoprazole or an optically active isomer thereof or a salt thereof, and a group which does not contain lansoprazole or an optically active isomer thereof or a salt thereof and contains total 0.2 to 200 parts by weight of magnesium oxide and magnesium hydroxide per 1 part by weight of lansoprazole or an optically active isomer thereof or a salt thereof.

20. The chewable tablet according to claim 19, wherein the weight ratio between magnesium oxide and magnesium hydroxide is 1:0.2 to 1:50.
